(19) United States
(12) Patent Application Publication
(10) Pub. No.: US 2007/0185465 A1
(43) Pub. Date: Aug. 9, 2007

(54) Personal urinal

(76) Inventors: James Albert Alexander Campbell, London (GB); Joyce Veronica Campbell, London (GB); Rushworth Oliver Charles Campbell, London (GB)

Correspondence Address: STITES & HARBISON PLLC, 1199 NORTH FAIRFAX STREET, SUITE 900, ALEXANDRIA, VA, 22314, US

(21) Appl. No.: 10/591,247

(22) PCT Filed: Jan. 27, 2005
(86) PCT No.: PCT/GB05/00290

§ 371 Date: Aug. 31, 2006

(30) Foreign Application Priority Data
Mar. 1, 2004 (GB) 0404603.3

(51) Int. Cl. A61F 5/44

(52) U.S. Cl. 604/347; 604/329

(57) ABSTRACT

A urinal device which has a groin shape urine collection bag, manufactured from impervious material which may be plastic. Preferably urine enters the groin bag through the upper edge, higher than the point of exist. A sheath appendage, or in the case of a female, a specially designed elliptical bowl receives urine or other body waste from the body assisted by a battery operated pump. The groin bag may be reusable or disposable, and conveniently emptied via the detachable self closing outlet. A liquid detector comprising two or more perforated steel mesh plates, preferably stainless steel may be provided with optional warning device. The urinal device may also be used as a gravity fed system with a conventional leg bag or freestanding container.

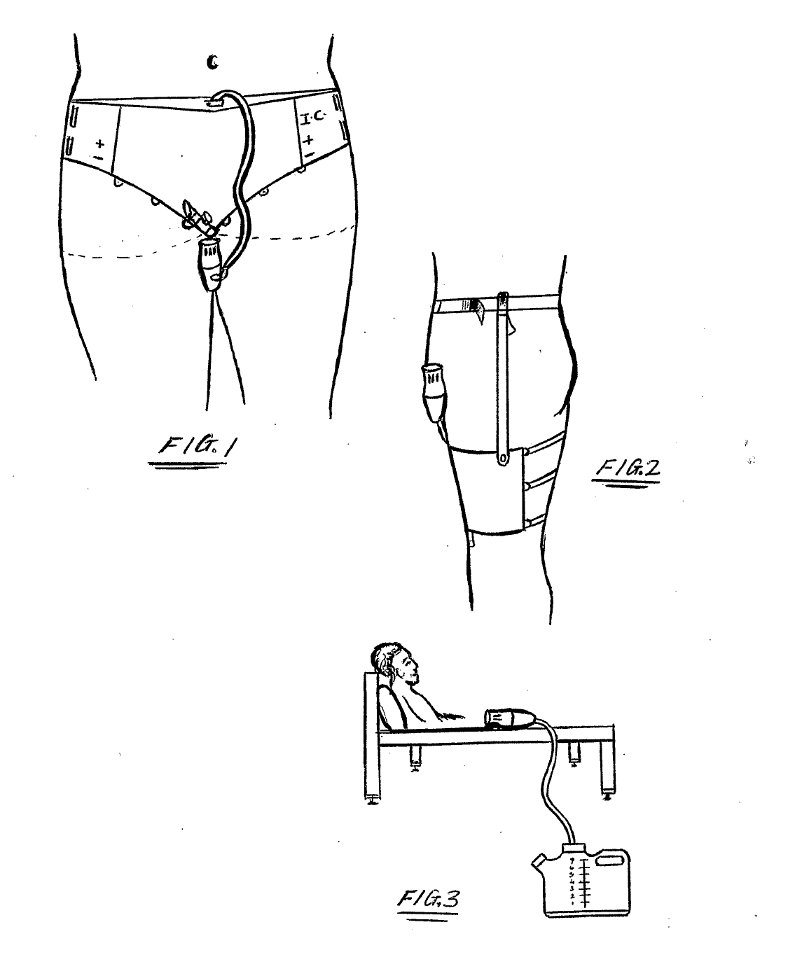

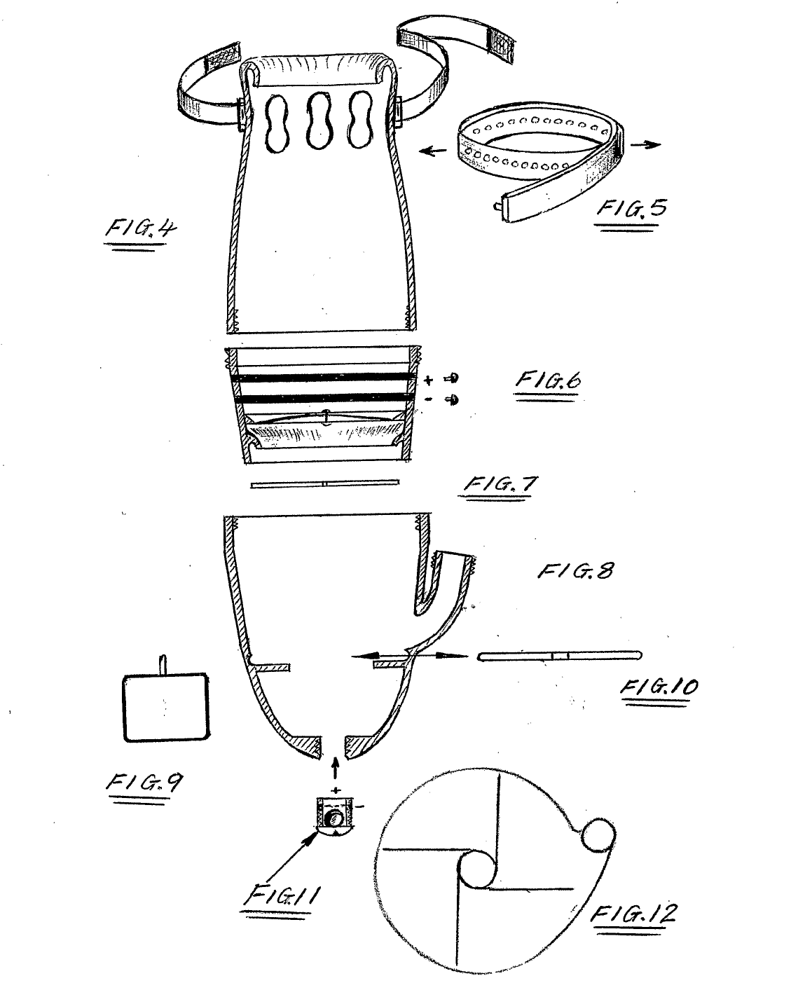

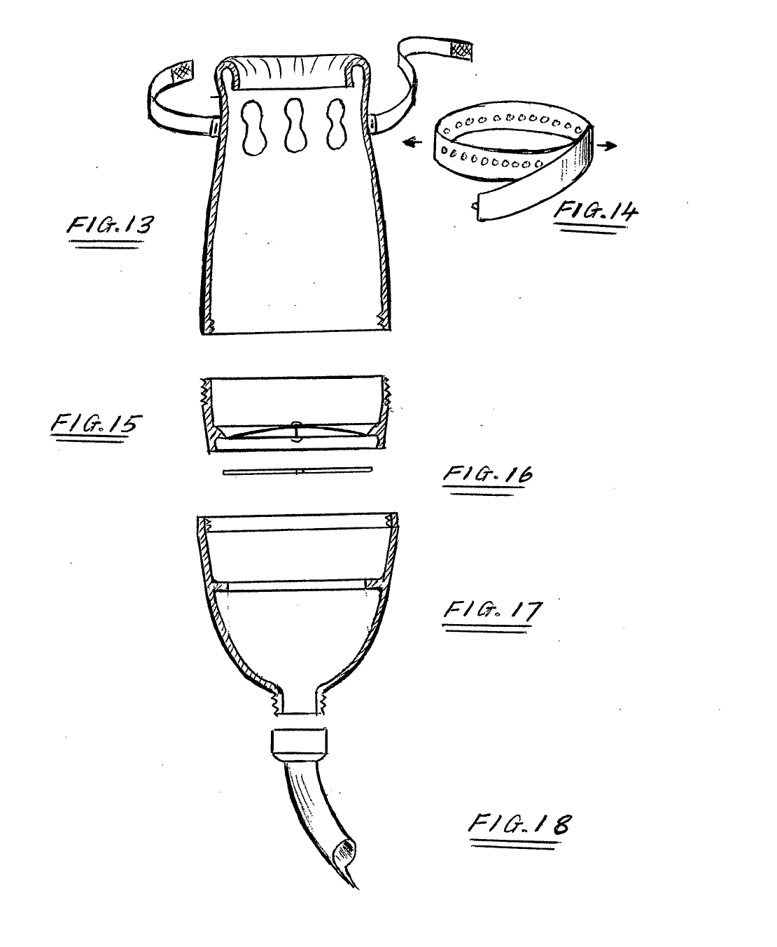

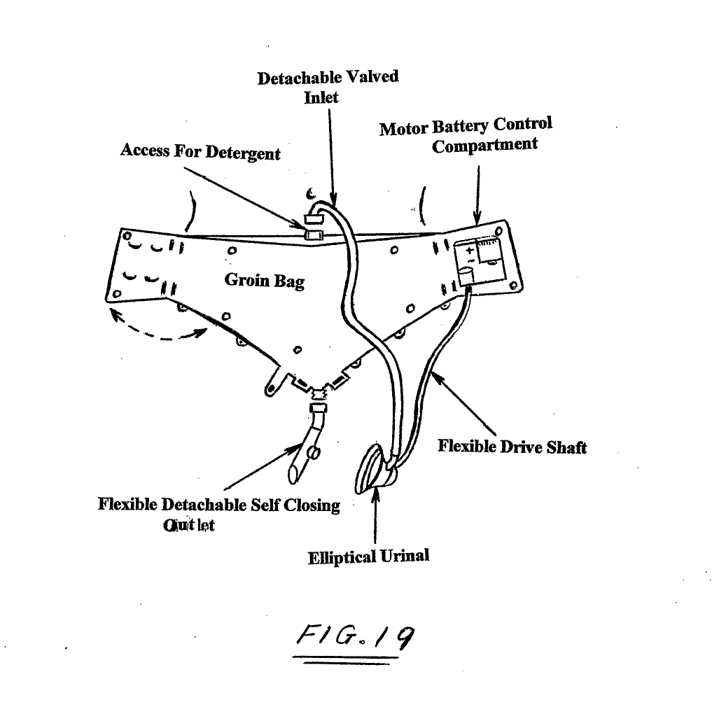

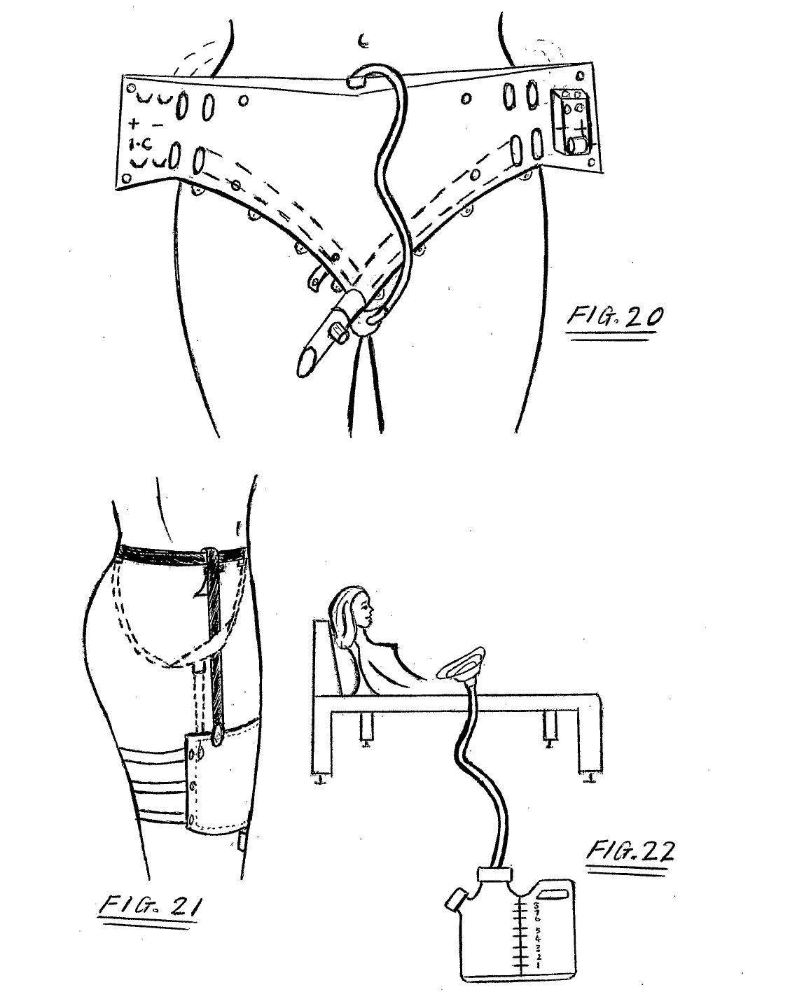

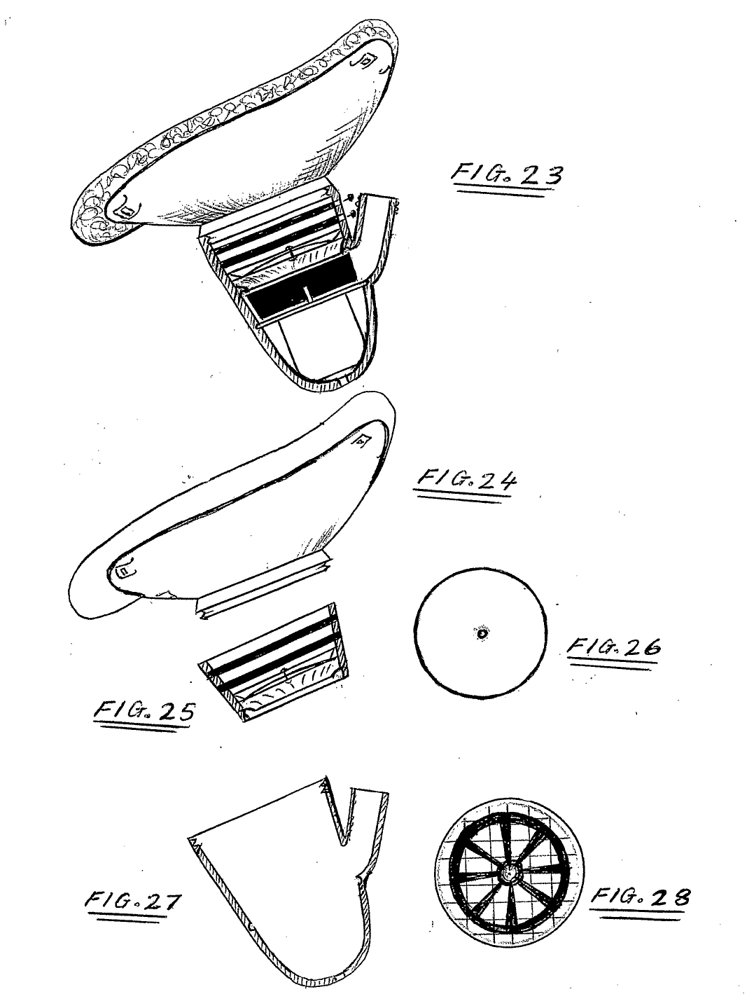

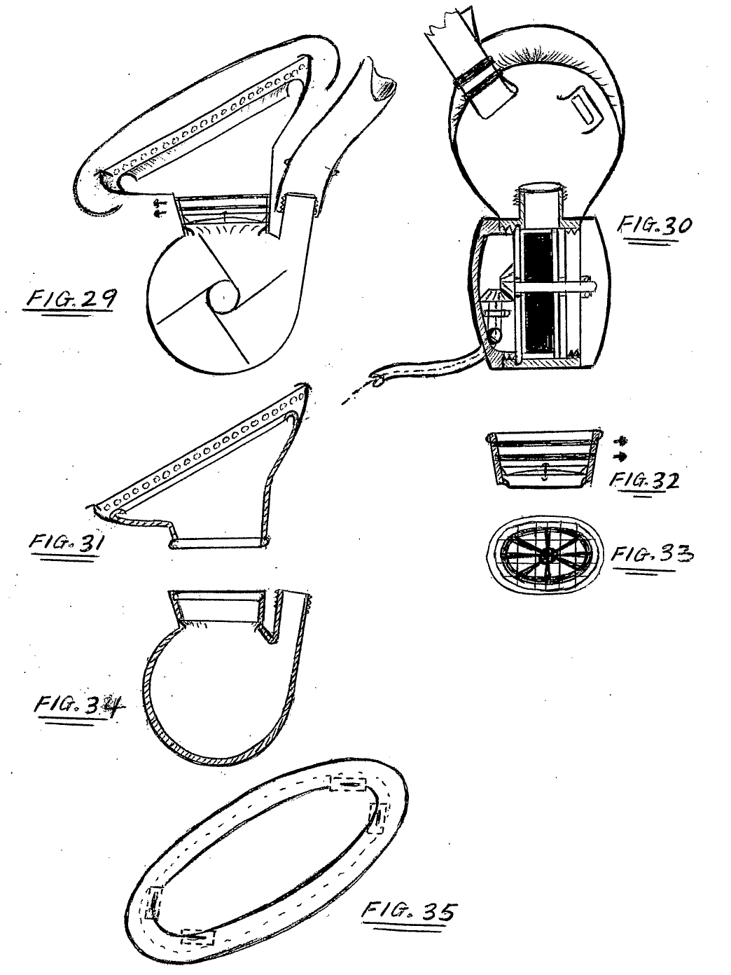

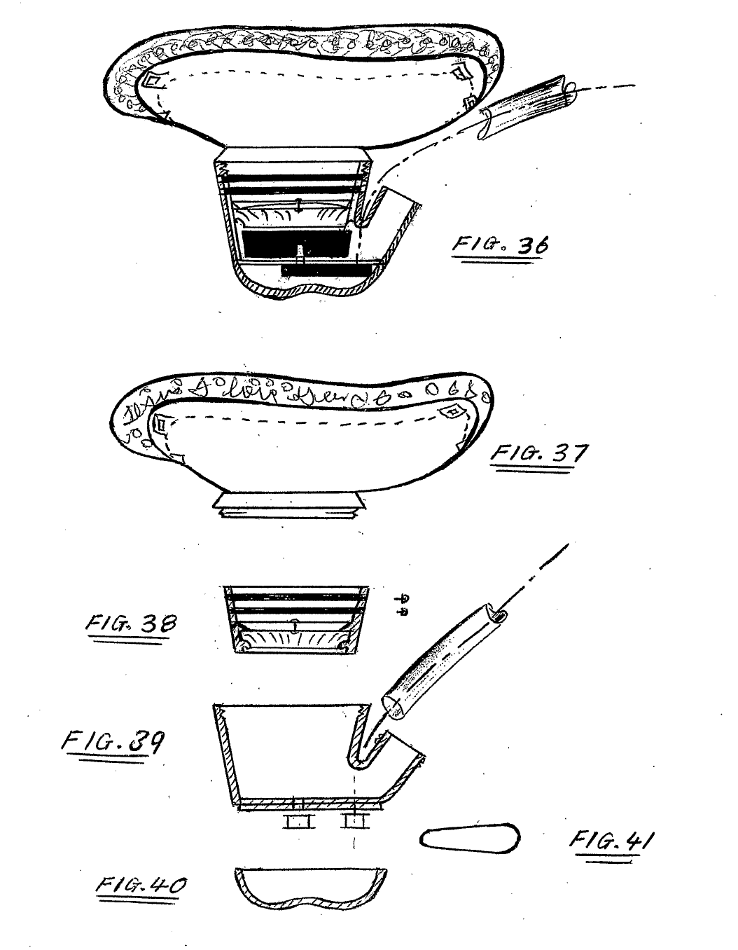

PAGE 1/11, shows three applications of the male configuration of the incontinence device, wherein FIG. 1 is a motorised application of the system. FIG. 2 a leg bag application of the system and FIG. 3 a free standing application of the system with calibrated container.

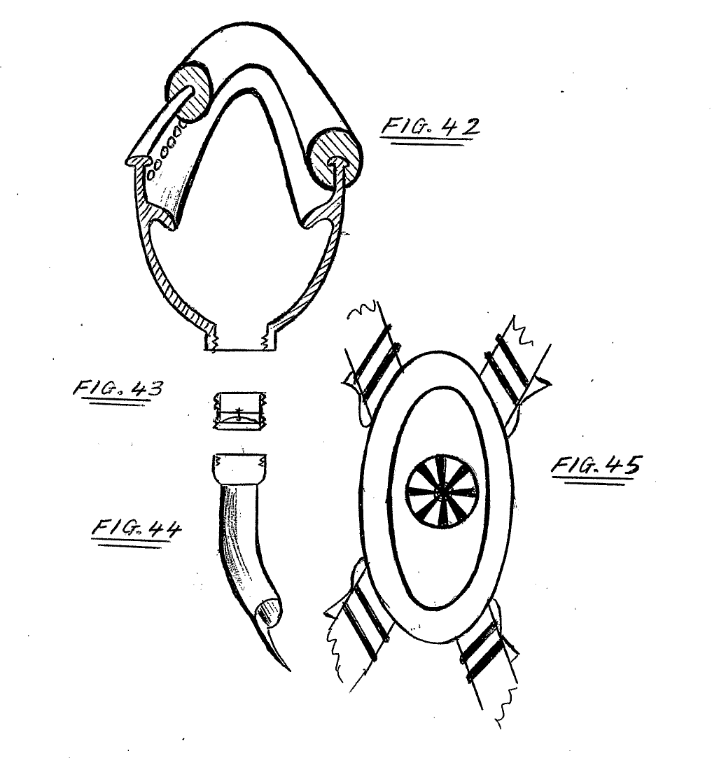

PAGE 2/11, shows a cross section through the dismantled male system wherein FIG. 4 is the vented appendage with internal rolled upper edge and low tension elasticated adjuster. FIG. 5 shows the expandable ring, FIG. 6 shows the lockable liquid detector with the non-return valve and circumferential anti-rise rim. FIG. 7 shows the removed flexible gland of the non-return valve. FIG. 8 shows the empty housing, FIGS. 9 shows the motor, FIG. 10 shows a press fit base plate, FIG. 11 is the removable leveling device and FIG. 12 is a plan revealing the fin/propeller position.

PAGE 3/11, shows a cross section through the male gravity fed system, wherein FIG. 13 is the vented appendage with internal rolled upper edge and low tension elasticated adjuster, FIG. 14 shows the expandable ring, FIG. 15 is a self-locking non-return valve, FIG. 16 shows the removed flexible gland of the non-return valve, FIG. 17 shows the empty housing and FIG. 18 is a detachable outlet that will lead to a leg bag or free standing container.

PAGE 4/11, FIG. 19 shows a labelled female configuration of the incontinence system.

PAGE 5/11, shows three applications of female configuration of the incontinence device wherein FIG. 20 is the motorised application of the system. FIG. 21 the gravity fed application of the system incorporating a leg bag. FIG. 22 the gravity fed application of the system with free standing container.

PAGE 6/11, FIG. 23 shows a cross section through the assembled direct drive system. FIG. 24 shows the removed elliptical bowl with the pervious membrane attached, FIG. 25 shows the lockable liquid detector with the non-return valve and circumferential anti-rise rim, FIG. 26 is a press fit base plate, FIG. 27 shows the empty housing and FIG. 28 is a plan of the non- return valve and liquid detector.

PAGE 7/11, FIG. 29 shows a cross section through the flexible drive system revealing the position of the impeller and the flexible, detachable outlet that will lead to the groin bag. FIG. 30 shows the system with the flexible drive shaft with 1 of 4 adjustable anchoring straps. FIG. 31 shows the cross section through the removed elliptical bowl with its elevated circumferential perforated edge. FIG. 32 and FIG. 33 show a section and plan of the non-return valve and the liquid detector with terminal screws. FIG. 34 shows the empty housing and FIG. 35 shows the removed pervious membrane.

PAGE 8/11, FIG. 36 shows a cross section through the assembled belt driven device. FIG. 37 shows the removed elliptical bowl with the pervious membrane. FIG. 38 shows the liquid detector and the non-return valve. FIG. 39 shows the empty housing, FIG. 40 shows the female profile base plate and FIG. 41 shows the removed belt.

PAGE 9/11, FIG. 42 shows a cross section through the gravity fed system, revealing the profile of the elliptical bowl with flexible detachable membrane, its elevated perforated edge and the slope of the internal circumferential rim. FIG. 43 is a non-return valve and FIG. 44 is a detachable inlet that will lead to a leg bag or a free standing container. FIG. 45 is a plan of the system revealing the 4 adjustable anchoring straps, the pervious membrane and the non return valve.

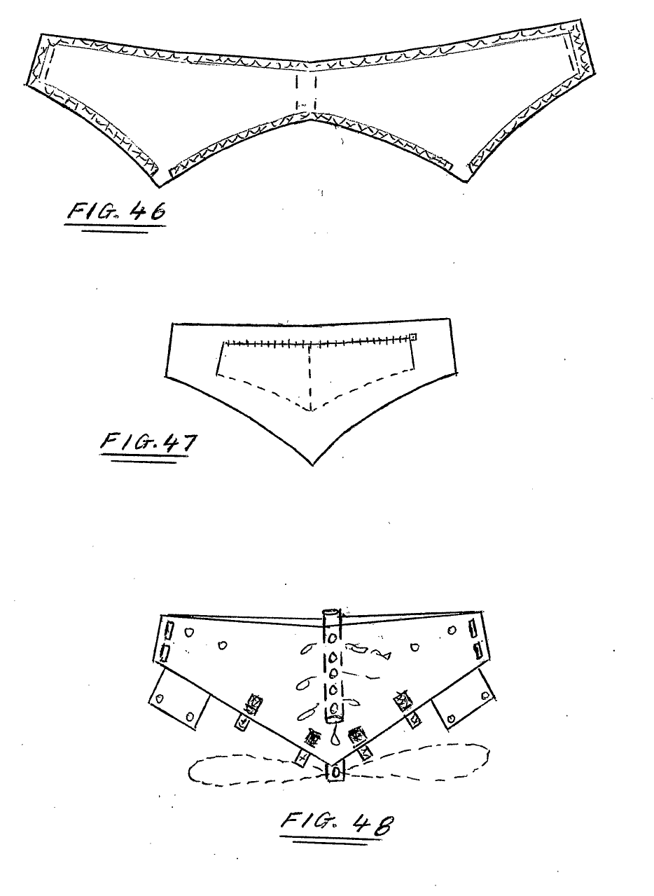

PAGE 10/11, FIG. 46 shows the development of the quilted reusable jacket. FIG. 47 shows the position of the 2 zippable compartments. FIG. 48 is a disposable groin bag with a perforated inlet to aid dispersion.

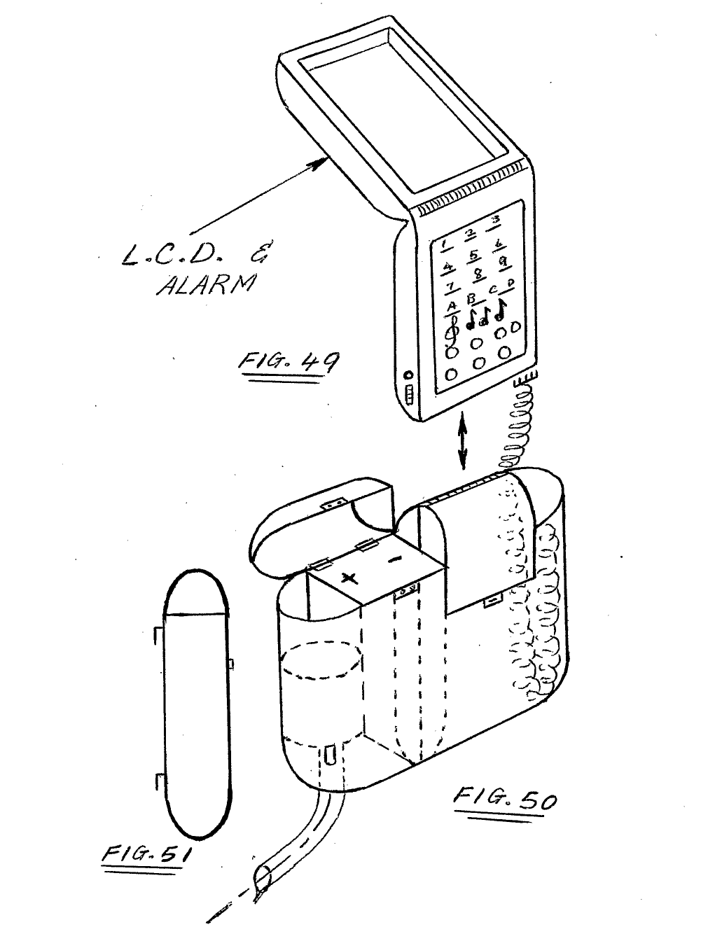

PAGE 11/11, FIG. 49 and FIG. 50 shows an isometric view of the clippable electronic system to be fitted inside the groin bag compartment, revealing the location of the motor, the flexible drive shaft, the battery, the visual L.C.D. unit and control panel enclosed and removed. FIG. 51 is the end view of the control unit.

1) An incontinence device for receiving urine from a user comprising a bag tailored to fit the groin region of the user wherein the bag accepts urine above the point of exit via a tube and has an additional flexible detachable self closing outlet wherein the bag has two compartments to hold a container housing a motor with flexible drive, battery and electronic circuit, a housing comprising a motorised system for accepting urine consisting of an automatic liquid detector a non return valve and a motorised pump.

2) An incontinence device according to claim 1, wherein the motorised housing is made of plastic with terminal screw receptacles, wherein said housing incorporates a removable vertical leveling device, wherein said device audibly alerts the user in the event of the motorised system/impeller unit becoming elevated to an extent that it is unable to accept urine (when sitting or lying) wherein said device is activated after a predetermined time (5 seconds).

3) An incontinence device according to claim 2, wherein the motorised housing is tapered in shape to allow the contents to be removed, cleaned and reassembled in one direction only.

4) An incontinence device according to claim 1 wherein the 360° sensitivity liquid detector and non return valve is made as one unit to aid easy dismantling and reassembling after cleaning, wherein the valve's gland can be extracted during menstruation thereby preventing sticking and the liquid detector can be electronically varied in sensitivity by the user to cater for personal need.

5) An incontinence device according to claim 3 wherein the motorised housing accommodates a flexible, detachable, expandable and vented appendage into which a penis can be inserted, wherein said appendage has an internally rolled upper edge to facilitate an expandable ring thereby catering for variation in size and an additional low tension elasticated band to permit minor expansion and contraction.

6) An incontinence device according to claim 5, wherein said detachable appendage permits safety overflow in the event of an electrical or mechanical malfunction.

7) An incontinence device according to claim 1, wherein a detachable, flexible valve urine inlet is connected to the motorised unit and the groin bag.

8) An incontinence device according to claim 1, wherein said bag is made of flexible, impervious, reusable material, or sealed disposable diaper type construction, wherein said diaper type accepts a perforated inlet to aid the equal dispersion of urine, wherein said diaper type is best used at bedtime and during menstruation.

9) An incontinence device according to claim 1, wherein a groin bag further incorporates an adjustable elasticised waist and leg bands, wherein said bag has an upper inlet, and a lower outlet onto which a spring loaded self closing outlet is connected to vacate urine from said bag via the fly of the trousers when necessary.

10) An incontinence device according to claim 9, wherein the groin bag can be fitted inside a soft quilted washable and reusable jacket to promote comfort, wherein said jacket has two zippable compartments to contain female menstrual paraphernalia.

11) An incontinence device wherein the motorised system/impeller unit is located directly below the point of exit, wherein all urine entering the system does so exclusively under gravity, and is then impelled along the flexible tube into the groin bag to be vacated when necessary.

12) An incontinence device according to claim 11, comprising a vertical weight bearing leg strap or a free standing calibrated container herein illustrated.

13) An incontinence device according to claim 1, wherein a motorised/impeller system can be either directly driven, flexibly driven incorporating a gear system, or flexibly driven incorporating a pulley system.

14) An incontinence device according to claim 1, wherein said device contains an elliptical shape urinal bowl made of plastic, with 4 external anchoring points onto which adjustable elasticated straps are placed to anchor the device onto the adjustable waistband of a female user.

15) A female incontinence device according to claim 14, wherein said elliptical bowl has a vertical and elevated circumferential perforated edge which accepts a soft, flexible, pervious, detachable, washable and reusable membrane.

16) An incontinence device according to claim 15, wherein said elliptical bowl permits safety overflow in the event of an electrical or mechanical malfunction.

17) An incontinence device according to claim 15, wherein said elliptical bowl has an internal circumferential rim, thereby preventing urine from reaching the pervious membrane, wherein said container can be fitted with a blood sensitive warning device.

18) An incontinence device according to claim 1 wherein the container, housing the motor with the flexible drive shaft, the electronic circuit, and battery are held in position within the groin bag flap/compartment.

19) An incontinence device according to claim 1, wherein the electronic device permits the user to select, program, customise and control the volume of all audible signals, and vary the speed of the motor to comply with individual demand.

20) An incontinence device according to claim 18, wherein said electronic device facilitate an audible or visual low voltage signal, wherein said device can be fitted with a mini-jack earphone to assist the hearing impaired.

21) An incontinence device according to claim 1, wherein said electronic device can incorporate a glucose sensitive detector with digital storage and read-back facility via an external monitor or the purpose built monitor V.D.U.

22) An incontinence device according to claim 17, wherein said electronic device incorporates a plug in P.C.B wherein said P.C.B. can be removed and upgraded to cater for changing demands.
